(19) United States
(12) Patent Application Publication
(10) Pub. No.: US 2012/0078085 A1
(43) Pub. Date: Mar. 29, 2012

(54) Method of Analysis for Dynamic Magnetic Resonance Perfusion Imaging

(75) Inventors: Hui Xue, Franklin Park, NJ (US); Marie-Pierre Jolly, Hillsborough, NJ (US); Xiaoguang Lu, West Windsor, NJ (US); Jens Gühring, Erlangen (DE)

(73) Assignee: Siemens Corporation, Iselin, NJ (US)

(21) Appl. No.: 13/246,910

(22) Filed: Sep. 28, 2011

Related U.S. Application Data
(60) Provisional application No. 61/387,488, filed on Sep. 29, 2010.

(51) Int. Cl. A61B 5/055; A61B 5/02

(52) U.S. Cl. 600/420

(57) ABSTRACT

A method (100) of processing myocardial MR perfusion images that corrects imaging errors arising from myocardial motion and B-1 field inhomogeneity (115-135); segments the myocardium images (140, 145); and calculates perfusion measures that enable analysis of the segmented myocardium images (150).

### CROSS-REFERENCE TO RELATED APPLICATIONS

This application claims the benefit of Provisional U.S. Patent Application Ser. No. 61/387,488 entitled, “Fully Automatic Dynamic Imaging Segmentation and Segmental Analysis”, filed in the name of Hui Xue, Marie-Pierre Jolly, Xiaoguang Lu, and Jens Guehring on Sep. 29, 2010, the disclosure of which is hereby incorporated by reference herein.

### FIELD OF INVENTION

The present invention relates to magnetic resonance (MR) imaging. More particularly, the present invention relates to dynamic magnetic resonance (MR) perfusion imaging.

### BACKGROUND OF THE INVENTION

Dynamic magnetic resonance (MR) perfusion imaging is a medical imaging approach that consists of acquiring a sequence of MR images of the heart after having injected the patient with a contrast agent. The goal of the technique is to measure the amount of contrast perfusion in the myocardium of the heart. The measurements are then used by health professionals to assess the clinical condition of the heart, specifically, the motion and functioning of the myocardium and myocardial blood flow.

The clinical routine to evaluate myocardial perfusion MR images is accomplished by a qualitative visual reading by a health professional and/or an analysis of computed myocardial perfusion quantitative maps from the pixel-wise or segmental perfusion signal intensity curves. FIG. 1 shows a basic perfusion signal intensity curve and associated perfusion parameters. For each pixel of a myocardial perfusion MR image, the signal-time curve may be analyzed and the perfusion parameters may be calculated. The parameters include, for example, up-slope (SLOPE), time-to-peak (TTP), peak time (PT) and area-under-curve between foot and peak (AUC, not specifically labeled). The graph notations t_{f} and tₚ are foot time and peak time, respectively. Typically, time may be measured in seconds and intensity in AU (arbitrary units).

Despite its proven clinical significance, certain technical challenges prevent dynamic MR perfusion imaging from being added to the clinical workflow, i.e., specified patient care activities. For example, complex cardiac motion caused by respiration, irregular heart rates, and/or imperfect cardiac gating may introduce errors or artifacts in the imaging. Another challenge is the overcoming of B1-field inhomogeneity caused by non-uniform characteristics of the receiver coils of the MR imaging system. While qualitative visual reading from dynamic MR perfusion imaging is often not compromised by this effect, B1-field inhomogeneity can result in errors of quantitative or semi-quantitative analysis, which aims to estimate perfusion parameters, such as up-slope (SLOPE), area-under-curve (AUC) or myocardial blood flow.

The long and labor-intensive analysis process that accompanies the technique is also an important obstacle to broad clinical utilization of dynamic MR perfusion imaging. Typically, clinical assessment of heart conditions requires sufficient coverage of the left ventricle (LV). However, due to the limited imaging efficiency of current MR scanners, a typical MR perfusion sequence acquires several two dimensional (2D) images to approximate the coverage or extent of the LV. A minimum of three short axis slices covering the basal, mid-ventricular, and apical portions of the LV is recommended. To maintain sufficient temporal resolution during the first-pass of the contrast bolus, each slice of the LV volume should be imaged every one to two heart beats for a total duration of approximately 60 seconds. As a result, a typical perfusion study can produce approximately 250 images. Further, to generate signal time-intensity curves in order to estimate perfusion parameters, manual delineation of the endocardium and epicardium is required due to cardiac motion noted above. This process is time-consuming, and can be even lengthier in patients who are unable to hold their breath to try to overcome respiration-induced cardiac motion and who require the MR image data acquisition to be performed in a free breathing fashion.

### SUMMARY OF THE INVENTION

The aforementioned problems are obviated by the present invention which provides a method of dynamic MR perfusion imaging, comprising obtaining a series of perfusion images of a target cardiac area; correcting imaging errors arising from cardiac motion; correcting imaging errors arising from surface coil inhomogeneity; segmenting the target cardiac area; and generating perfusion parameters for the perfusion images that enable analysis of the segmented target cardiac area. The target cardiac area may comprise the myocardium. The perfusion images may be first-pass perfusion images. The correcting imaging errors arising from cardiac motion may comprise detecting a key-frame in the perfusion series. In such case, the key-frame may define a reference image and the correcting step may comprise correcting the relative motion between the reference image and other phases. Further, the key-frame may comprise an image frame in which the target cardiac area has good contrast compared to the blood pool and surrounding tissues. Alternatively, the detecting step may comprise computing the standard deviation of intensity image for the perfusion series and selecting the frame having similar contrast to the standard deviation of intensity image as the key-frame. In such case, the selecting step may comprise computing cross correlation ratios (CC) between every phase in the perfusion series and the standard deviation of intensity image and selecting the phase corresponding to the maximal CC ratio as the key-frame.

The correcting imaging errors arising from cardiac motion may further comprise performing a registration of the key-frame with other images in the perfusion series. In such case, the performing step may comprise performing consecutive motion compensation; consecutively performing multiple 2D-2D registrations between temporally adjacent images, or applying a fast variational non-rigid algorithm to the perfusion series.

The correcting imaging errors arising from surface coil inhomogeneity may comprise estimating a smoothing surface coil inhomogeneity field from proton density (PD) images of the target cardiac area and applying the estimated field to all perfusion frames. The estimating step may comprise acquiring the PD images before obtaining the perfusion series and estimating the surface coil inhomogeneity field via an approximation of B-Spline Free Form Deformation (FFD).

The segmenting step may comprise detecting selected cardiac landmarks. Alternatively, the segmenting step may comprise detecting the center of the blood pool and the right ventricle insertion points as cardiac landmarks. In such case, the segmenting step may further comprise segmenting the endocardium and the epicardium contours and, also, the method may further comprise splitting the epicardium and endocardium contours according to the American Heart Association (AHA) standards model for an analysis of the segmented target cardiac area. The generating perfusion parameters may comprise estimating perfusion parameters for each pixel in a respective image.

The present invention also provides a computer-assisted method of processing myocardial MR perfusion images, comprising correcting imaging errors arising from myocardial motion and B-1 field inhomogeneity; segmenting the myocardium images into standards-defined segments; and calculating perfusion measures that enable analysis of the segmented myocardium images. The correcting step may comprise detecting a key-frame of the perfusion images and performing a registration between the key-frame and other phases of the perfusion images to correct the relative motion between the key-frame and a respective phase. The performing step may comprise performing consecutive motion compensation on the perfusion images. The correcting step may also comprise applying an estimated smoothing B-1 field imhomogeneity field to all perfusion frames to correct for B-1 field coil inhomogeneity. Alternatively, the correcting step may comprise estimating B-1 field-induced intensity variations in a respective image and generating an estimated B-1 field-induced intensity variation-compensated image.

The segmenting step may comprise detecting cardiac landmarks and segmenting the epicardium and endocardium contours. The segmenting the epicardium and endocardium contours may comprise classifying the pixels in the perfusion images; transforming the key-frame image into a polar image based on the detected cardiac landmarks; computing gradients, in the polar space, for the recoveries of the epicardium and endocardium contours; and recovering each of the epicardium and endocardium contours by finding the respective shortest path in the polar image. The classifying step may comprise extracting the main gray level modes in the key-frame image. The transforming step may comprise determining a polar coordinate system in which to transform the image by using the blood pool landmark as the center and the distance to the anterior right ventricle insertion point as a radius. The segmenting step may further comprise recovering the contours in a next slice by repeating steps a, b, and c; propagating the respective contours from the current slice to generate respective a priori contours for each of the epicardium and endocardium; and recovering each of the epicardium and endocardium contours by finding the respective shortest path in the polar image using the respective a priori contours.

The calculating step may comprise calculating perfusion parameter maps with estimated perfusion parameters for each pixel in an image or computing the American Heart Association (AHA) standards model for an analysis of the segmented myocardial images.

The present invention also provides a system for dynamic MR perfusion imaging, comprising an MR imaging system having an imager that images the cardiac area to acquire image data and a processor that manipulates the acquired image data and stored image data to correct imaging errors arising from myocardial motion and B-1 field inhomogeneity; segment myocardium images into standards-defined segments; and calculating perfusion measures that enable analysis of the segmented myocardium images.

### DESCRIPTION OF THE DRAWINGS

For a better understanding of the present invention, reference is made to the following description of an exemplary embodiment thereof, and to the accompanying drawings, wherein:

FIG. 1 shows a perfusion signal intensity curve and associated perfusion parameters;

FIG. 2 is an MR imaging system (simplified) that performs MR imaging in accordance with the present invention;

FIG. 3 is a flow chart of a method of MR imaging carried out in accordance with the present invention;

FIG. 4 is an illustration of consecutive motion compensation;

FIG. 5 is an illustration of motion compensation of a free-breathing perfusion series;

FIG. 6a shows a proton density cardiac image acquired before normal perfusion acquisition;

FIG. 6b shows an intensity profile across a heart region of FIG. 6a;

FIG. 7a shows the estimated bias field for the PD image in FIG. 6a using the method of FIG. 3;

FIG. 7b shows the corrected intensity profile for the PD image in FIG. 6a using the method of FIG. 3;

FIG. 8 is a flow chart of a segmentation method carried out in accordance with the present invention;

FIG. 9a shows a perfusion time series for a multi-scale perfusion parameter estimation;

FIG. 9b shows a perfusion signal intensity curve for a selected point in FIG. 9a; and

FIG. 10 shows segmental analysis images for cardiac perfusion MR imaging.

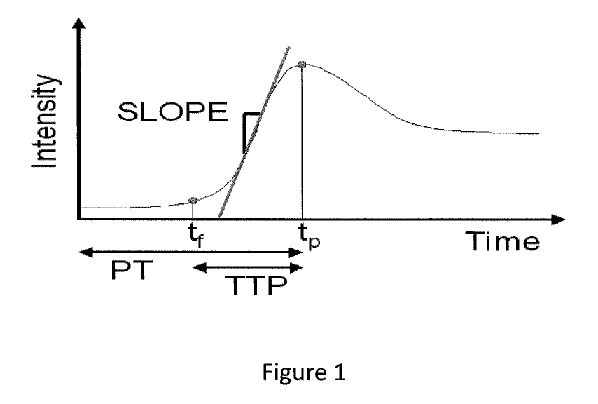

### DETAILED DESCRIPTION

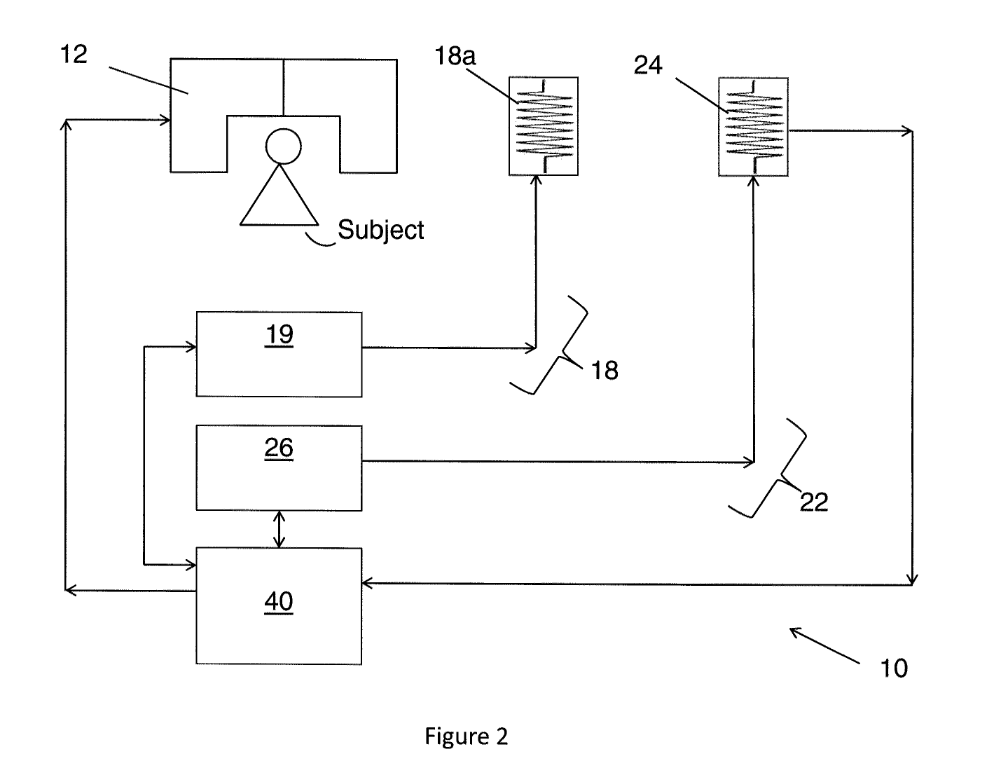

FIG. 2 is a block diagram of a conventional MRI scanner 10 (simplified) that performs myocardial perfusion MR imaging in accordance with the present invention. A main magnet 12 generates a strong static magnetic field in an imaging region where the subject (i.e., patient) is introduced. The magnet 12 is used to polarize the target cardiac area, i.e., certain atoms in the target cardiac area that were previously randomly-ordered become aligned along the magnetic field. A gradient coil system 18, having a gradient coil subsystem 18a and a gradient coil control unit 19, generates a time-varying linear magnetic field gradient in respective spatial directions, x, y and z, and spatially encodes the positions of the polarized or excited atoms. An RF system 22, having an RF coil subsystem 24 and a pulse generation unit 26, transmits a series of RF pulses to the target cardiac region to excite the “ordered” atoms of the target cardiac area. The RF coil subsystem 24 may be adapted to switch between a transmission mode and receiver mode.

A control or computer system 40 coordinates the pulse generation unit 26, the gradient coil control unit 19, and other components to carry out a desired MR image pulse sequence. The scanner 10 repeats the MR image pulse sequence a number of times so the atoms oscillate around the polarized alignment direction (along the main magnetic field) during the excited state caused by the energy of RF pulses. The atoms release the RF energy, i.e., generate an RF signal, during the resonance or oscillation and as the atoms return to their respective alignments. The RF coil subsystem 24 receives or detects the released RF energy and generates spatially-coded MR signals to the computer system 40. It is noted that the subject may be injected with contrast agent that permeates the target cardiac area in order to assist in the capture of image data and the resulting image visualization.

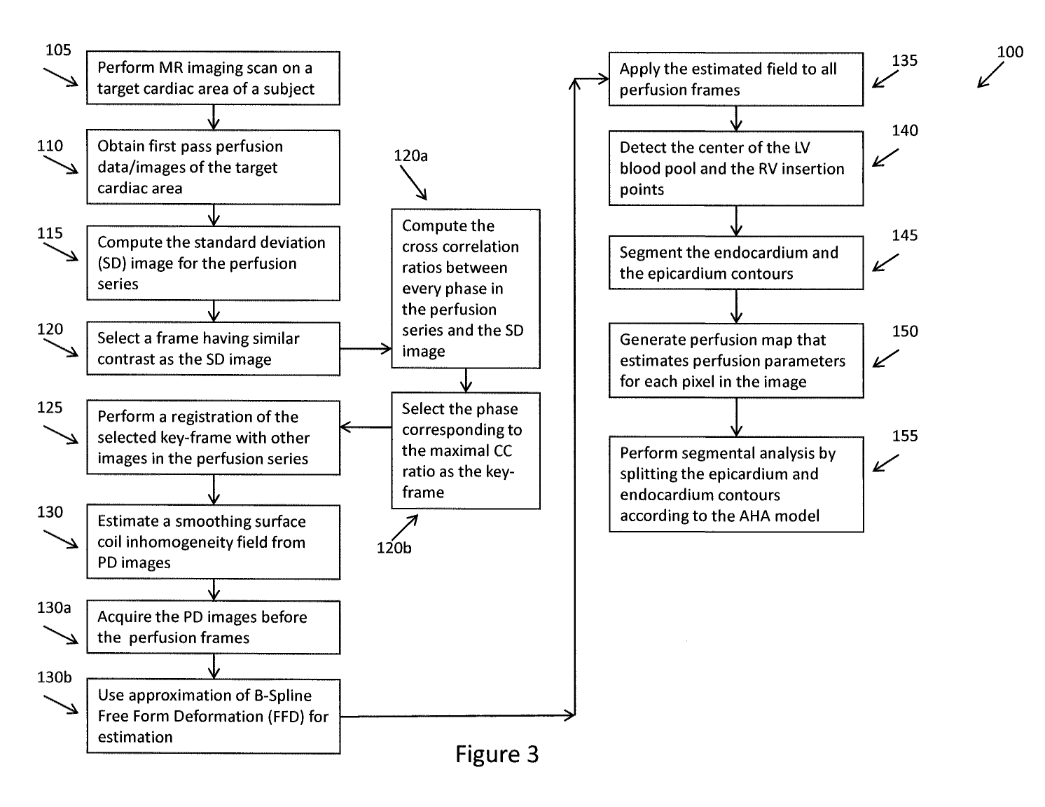

The computer system 40, which controls the operation of the MR scanner 10 and its components, processes the MR signals to transform them into a visual representation of the target cardiac region (i.e., reconstructed MR images) for display, storage, image processing, and/or other usage. The MRI scanner 10 and, in particular, the computer system 40, is adapted to permit the imaging scanner 10 to operate and to implement methods of the present invention, for example, as shown in FIG. 3.

Generally, the present invention provides methods that automatically correct errors arising from the myocardium motion and the B-1 field inhomogeneity, segment the myocardium into the 16 or 17 segments of the American Heart Association (AHA) standards model, and calculate perfusion measures such as the upslope, time to peak, peak time, and area under the curve of the perfusion signal for each segment as well as for every pixel. The methods broadly comprise the steps of key-frame detection; consecutive motion compensation; surface coil inhomogeneity correction; landmark detection; myocardium segmentation; and segmental analysis and parametric map calculation. The methods provide novel processing for cardiac perfusion MR imaging that overcomes some of the drawbacks in using the technique described above.

FIG. 3 shows a flow chart of a method 100 of MR imaging carried out in accordance with the present invention. A health professional operates the MRI scanner 10 to perform an imaging scan of the target cardiac area by implementing an MR perfusion pulse sequence or series (Step 105). The pulse sequence is designed to carry out myocardial perfusion MR imaging. The MRI scanner 10 then obtains the first-pass perfusion data/images of the target cardiac area (Step 110). To correct for the myocardium motion, the method 100 initially aims to detect a key-frame in the images for the perfusion series. This key-frame is defined as the reference image, and relative motion between other phases (i.e., the same image but at the different time-points in the scanning cycle for a slice) and this reference image will be corrected by the method 100. To improve the motion compensation, this key-frame should be a frame in which the myocardium has good contrast compared to the blood pool and surrounding tissues. The method 100 uses a key-frame selection approach which is based on the observation that during the contrast uptake (i.e., the absorption and retention of the contrast) the image intensity in regions where the contrast bolus enters will have higher standard deviation (SD) along the time dimension (of a perfusion signal intensity curve). Accordingly, the MRI scanner 10 first computes the standard deviation image for the perfusion series (Step 115). Although inconsistent myocardial motion can degrade the sharpness in the imaging of myocardium, the contrast between myocardium and surrounding tissues in the SD image is found to be consistently noticeable. This observation holds true for the described perfusion MR pulse sequences. The MRI scanner 10 then selects a frame having similar contrast as the SD image as the key-frame (Step 120). For this purpose, the cross correlation ratios (CC) between every phase in the perfusion series and the SD image are computed (Step 120a). During the passing of contrast bolus, the CC ratio continues to increase and reaches its peak around the time point where the myocardium blood perfusion is maximized. The MRI scanner 10 therefore picks the phase corresponding to the maximal CC ratio as the key-frame (Step 120b).

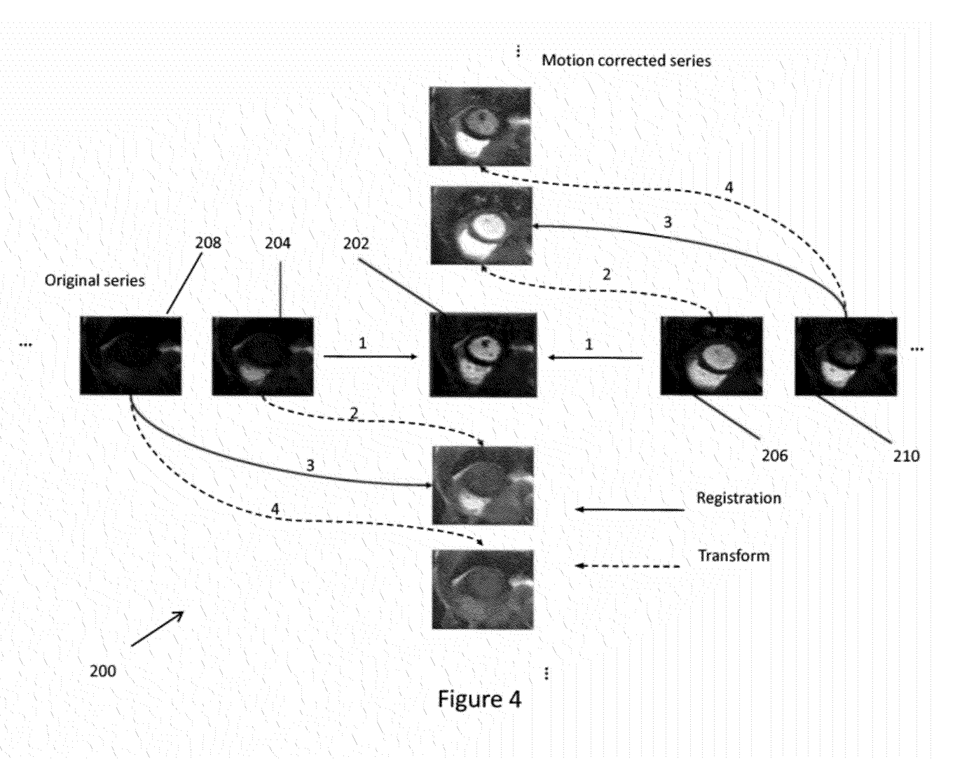

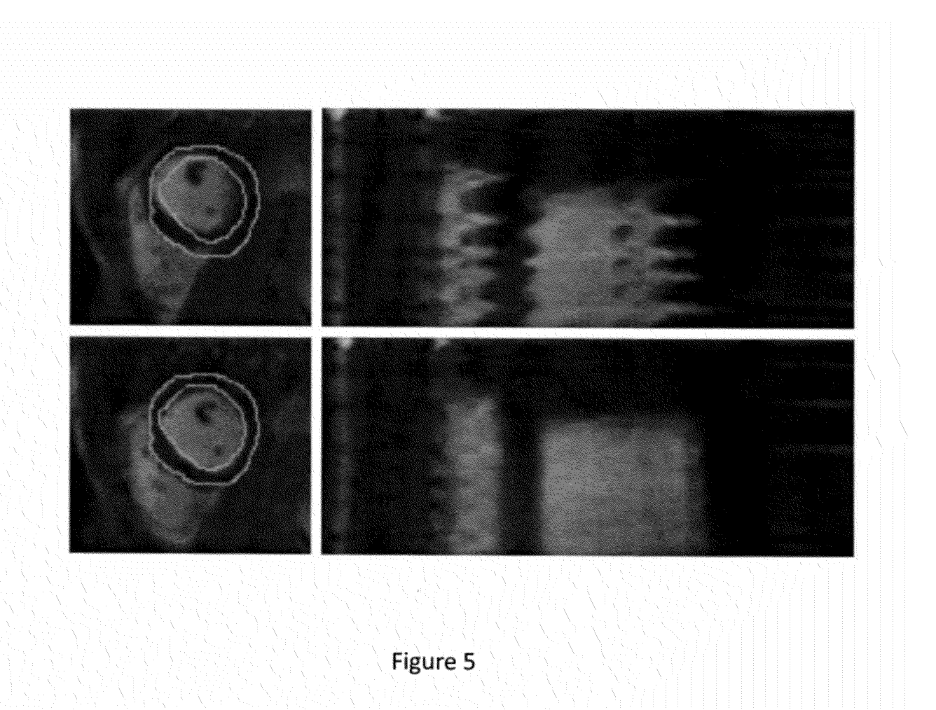

The MRI scanner 10 then performs a registration of the selected key-frame with other images in the perfusion series (Step 125). Based on the observation that registration is more robust if two slices to be aligned have similar contrast, the method 100 uses consecutive motion compensation to improve the performance of registration of the selected key-frame with the other images. FIG. 4 is an illustration of consecutive motion compensation 200. Motion compensation starts from the key-frame 202 and its direct image neighbors in the original perfusion series (i.e., the previous image 204 and the following image 206). After the first registration between the key-frame 202 and its direct image neighbors 204, 206 is finished (arrow 1), the direct image neighbors 204, 206 are each transformed into the key-frame coordinate system (arrow 2). The next previous image 208 and the next following image 210 are each registered to its respective warped neighbor 204, 206 (arrow 3) that has been transformed into the key-frame coordinate system—they are transformed themselves into the coordinate system of the respective warped neighbor 204, 206 (arrow 4). This process continues so every image is aligned to its transformed neighbor. In this scheme, registration is performed between two perfusion phases with similar contrast. The complete perfusion series is corrected by consecutively performing multiple 2D-2D registrations between temporally adjacent slices. Considering the temporal resolution of perfusion studies is usually one to two heart beats, adjacent frames consistently show similar contrast, even during the first pass of contrast agents. FIG. 5 illustrates motion compensation of a free-breathing perfusion series. In the first row of the figure, a TrueFISP 2D slice is overlaid with the myocardium contour of the key-frame followed by the associated intensity-time profile. In the second row of the figure, the same slice is shown after motion compensation followed by a corrected intensity-time profile (having the time scale on the y-axis).

A fast variational non-rigid registration algorithm may be applied by the MRI scanner 10 as the working-engine of perfusion motion compensation (such an algorithm is described further in an article by C. Chefd'hotel, G. Hermosillo, and O. Faugeras, entitled “Flows of Diffeomorphisms for Multimodal Image Registration”, Proc. IEEE Int. Symp. Biomedical Imaging: From Nano to Macro, 2002, Washington, D.C., USA, which is incorporated by reference herein). This approach can be considered as an extension of the classic optical flow method. In this framework, a dense deformation field is estimated as the solution to a calculus of variation problem, which is solved by performing a compositional update step corresponding to a transport equation. The regularization is added by low-pass filtering the gradient images which are in turn used as velocity field to drive the transport equation. To speed up the convergence and avoid local minima, a multi-scale image pyramid is created. The local cross correlation may be selected as the image similarity measure, as its explicit derivative can be more efficiently calculated than mutual information and still be general enough to cope with intensity fluctuation and imaging noise between two adjacent perfusion frames.

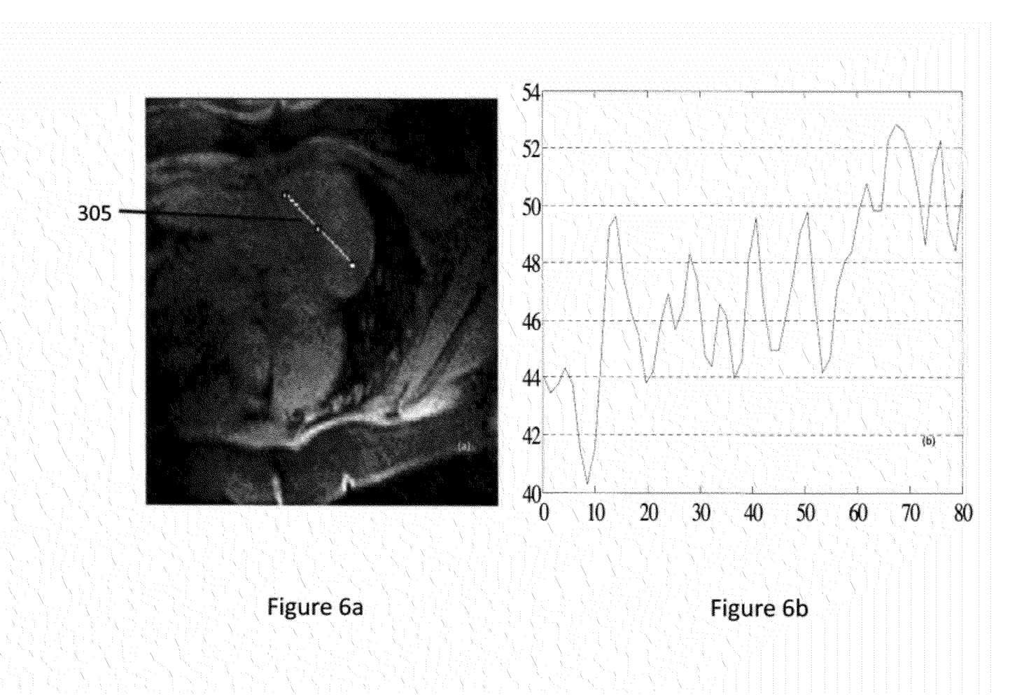

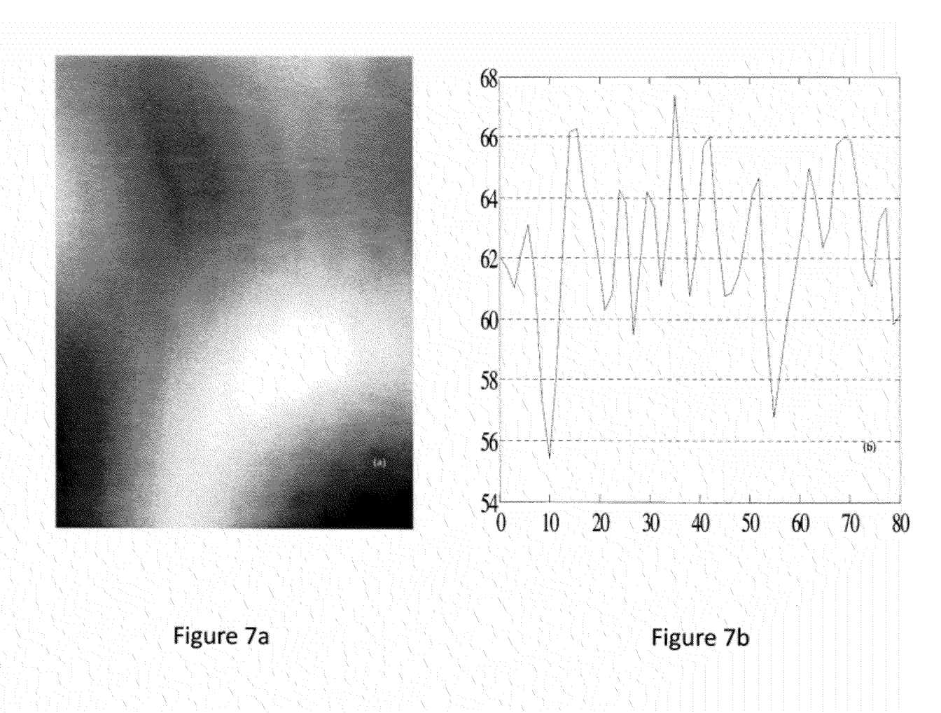

As noted above, the B1-field (or surface coil) inhomogeneity causes signal intensity variation which will affect quantitative assessment and must be corrected before computing perfusion quantitative maps. To correct for the surface coil inhomogeneity in the perfusion images, the MRI scanner 10 estimates a smoothing surface coil inhomogeneity field from proton density (PD) images (Step 130) and applies the estimated field to all perfusion frames (Step 135). FIG. 6a shows a PD image of the heart acquired before the normal perfusion acquisition. A PD image is produced by controlling the selection of MR scan parameters to minimize the effects of T1 and T2, resulting in an image dependent primarily on the density of protons in the imaging volume. FIG. 6b shows an Intensity profile (i.e., perfusion signal intensity) across the heart region 305 indicated in FIG. 6a. The intensity bias can be clearly observed in FIG. 6a. The MRI scanner 10 acquires a small number of PD images before the perfusion series acquisition (Step 130a) and, generally, estimates the surface coil inhomogeneity fields via the approximation of B-Spline Free Form Deformation (FFD) (Step 130b). The B-Spline FFD parameters are iteratively optimized by interleaving the tissue classification and surface coil inhomogeneity field calculation using Expectation-Maximization (EM) algorithm. This technique is more fully described in a previously-filed U.S. patent application, U.S. Ser. No. 13/031,321, by Hui Xue, Jens Guehring, and Sven Zuehlsdorff, entitled “Direct and Indirect Surface Coil Correction for Cardiac Perfusion MRI” filed on Feb. 21, 2011, which is incorporated by reference herein. FIG. 7 shows the estimated bias field (FIG. 7a) and the corrected intensity profile (FIG. 7b) for the PD image in FIG. 5. The estimated bias field is used to correct the entire perfusion time series.

The method 100 segments the myocardium in continuing the processing of the MR imaging. The MRI scanner 10 extracts the center of the blood pool when the contrast agent reaches the left ventricle (LV) center and extracts right ventricle (RV) insertion points (i.e., where the RV connects to the LV) when the contrast agent reaches the right ventricle (Step 140). This technique is more fully described in U.S. patent application, U.S. Ser. No. 13/234,694 by Bogdan Georgescu, Christoph Guetter, Jens Gühring, Marie-Pierre Jolly, Arne Littmann, Xiaoguang Lu, Hui Xue, and Sven Zuehlsdorff, entitled “Simultaneous Detection of Landmarks and Key-frames in Cardiac Perfusion MRI Using a Joint Spatial-Temporal Model” filed on Sep. 16, 2011, which is incorporated by reference herein. Generally, a spatial-temporal model is trained to recognize the above key landmarks using probabilistic boosting tree classifiers combined using marginal space learning to reduce the search space.

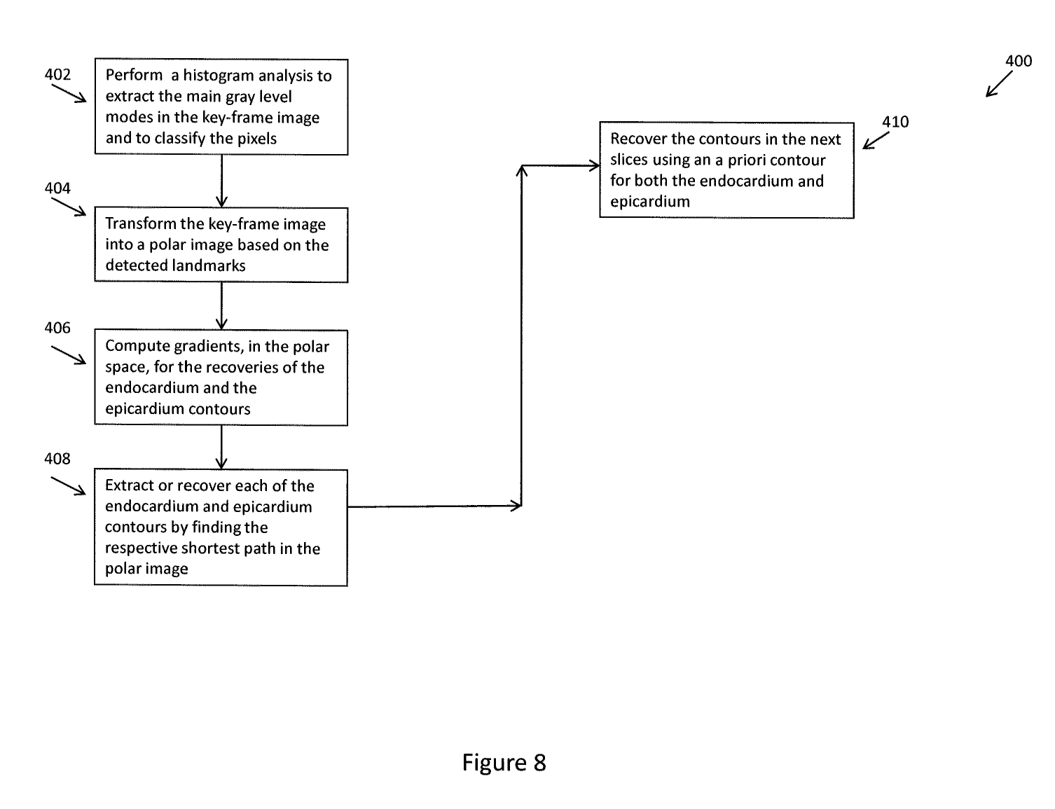

The method 100 then segments the myocardium by segmenting the endocardium and the epicardium contours (Step 145). This approach is similar to the single image segmentation described in an article by M-P. Jolly, C. Guetter, and C. Guehring, entitled “Cardiac segmentation in MR cine data using inverse consistent deformable registration”, Proc. Int. Symp. Biomedical Imaging: From Nano to Macro, April 2010, Rotterdam, The Netherlands, which is incorporated by reference herein. Since the perfusion sequence has been motion-corrected, a respective contour only needs to be recovered once. It will then appear at the same position on all phases. The segmentation method 400 comprises several steps that are carried out by the MRI scanner 10 and are shown in a flow chart in FIG. 8. The first step is to perform a histogram analysis to extract the main gray level modes in the key-frame image (Step 402). This permits the pixels to be preclassified. The image regions of interest are lungs, myocardium, left ventricle blood pool, and right ventricle blood pool. The classification of a pixel is based on the maximum gray level in the sequence and the frame at which the maximum happens. Once the main modes have been extracted in the histogram, the pixels may be classified using multi-fuzzy connectedness as proposed by the article by G. T. Herman and B. M. Carvalho, entitled “Multiseeded segmentation using fuzzy connectedness,” IEEE Trans. Pattern Analysis and Machine Intelligence, 2001, pp. 460-474, vol. 23, no. 5, which is incorporated by reference herein. The new labeled regions are then used to calculate probability distributions for each of the classes.

In a polar transformation step, the segmentation method 400 uses the blood pool landmark as the center and the distance to the RV insertion point (the anterior RV insertion point being the common clinical reference point used) as a radius to determine a polar coordinate system in which to transform the image (Step 404). In the polar image, the blood pool is at the top of the image, the myocardium is a horizontal band across the image, and the background is at the bottom. The endocardium and epicardium contours are expected to be recovered as two contours from the left to the right of the polar images.

The segmentation method 400 then computes gradients in the polar space (Step 406). Specifically, the MRI scanner 10 may compute the gradient for the recovery of the endocardium contour as a linear combination of the gradient of the maximum gray level image; the gradient of the response to the myocardium probability density; and the smooth image of the local transitions from LV blood to another label below in the pre-labeled image. The MRI scanner 10 may also compute the gradient for the recovery of the epicardium contour as a linear combination of the gradient of the first image in the sequence; the gradient of the last image in the sequence; the gradient of the response to the myocardium probability density; and the smooth image of the local transitions from myocardium to RV blood labels in the pre-labeled image.

Once the gradients have been computed, the cost function is defined as Energy=1/(Gradient²+Epsilon) where Epsilon is a small constant (typically 0.05) to bound the cost function. The segmentation method 400 then extracts or recovers the endocardium and epicardium contours by finding the shortest path using, for example, Dijkstra's algorithm in the polar image (Step 408). The pixels on the left side of the image may be defined as the starting points on the path and the pixels on the right side of the image may be defined as the ending points on the path. The branch and bound algorithm proposed by the article by B. Appleton and C. Sun, entitled “Circular shortest paths by branch and bound,” Pattern Recognition, 2003, pp. 2513-2520, vol. 36, no. 11 (which is incorporated by reference herein) may be used to guarantee that the respective contour is closed when converted back to Cartesian space. The recovery of the myocardium consists of three passes of Dijkstra's algorithm to:

1. recover the endocardium contour on its own;

2. recover the epicardium contour constrained by the endocardium contour; and

3. recover the endocardim contour constrained by the epicardium contour.

To recover the contours in the next slices, the segmentation method 400 propagates the respective contour from the current slice, for example, by finding the deformable registration between the maximum gray level image of the previous slice and the maximum gray level image of the current slice. This generates an a priori contour for both the endocardium and the epicardium (Step 410). Then, the gradient images are computed as previously described and the Dijkstra's algorithm is applied in three passes to recover the endocardium and epicardium contours. Only this time, the respective prior contour from the deformable registration is combined in the energy function (same as a cost function) in the form of a distance function by: Energy′=min(Energy+DistMap²/2,1/Epsilon). This maximizes a recovery of the best contour.

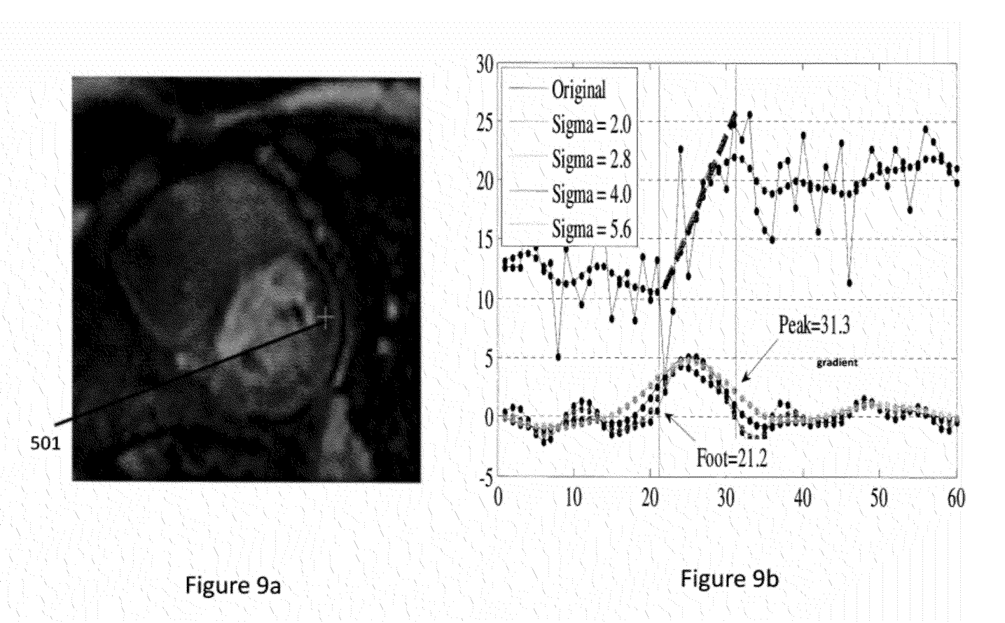

As a final step of the processing of the MR imaging, the method 100 calculates perfusion parameter maps and enables analysis of the segmented myocardium. Instead of computing perfusion parameters for every myocardial segment, the method 100 uses a robust map generation algorithm based on the scale-space theory, which estimates perfusion parameters for each pixel in the image (Step 150). The algorithm, using a signal intensity (SI) curve s(t), generates a series of smoothed curves sᵢ(t) by convolution with Gaussian kernels Sᵢ(t)=s(t)*gᵢ(t), where gᵢ(t) is a Gaussian function with the variance being τᵢ, I=1, . . . N and τᵢ<τⱼ,0<i<j<=N. Similarly, the algorithm computes the first-order derivative Sᵢ′(t) by convolution with the derivative of the Gaussian kernel. As no segment averaging is performed, the pixel-wise signal intensity curve s(t) can be quite noisy. This is shown in FIGS. 9a and 9b which together illustrate a multi-scale perfusion parameter estimation. FIG. 9a specifically shows a perfusion time series with clear perfusion deficit and FIG. 9b shows the SI curve for a selected point in FIG. 9a (marked by the cross 501) and detected contrast uptake. Although the drastic fluctuation due to imaging noise is clear from the SI curve, the multi-scale strategy implemented by the algorithm is able to detect the first uptake of contrast bolus.

To obtain a robust detection of first pass contrast bolus uptake, the algorithm only keeps the stable features that consistently appear across the whole scale space. As the first step, all local maxima and zero-crossings of gradient sᵢ′(t) are found as curve feature points. For each feature point, its appearance across all scales is checked using the concept of non-maximum suppression. The stable maximum with largest gradient is picked as the time-point corresponding to maximal up-slope. The foot time t_{f} is defined by the 20% of the maximal gradient, while the peak time tₚ is determined by the first stable zero-crossing point after the maximal up-slope, corresponding to the maximal intensity of first bolus uptake. Once the bolus uptake region is found, a weighted least-square fit is applied to this part of time-intensity curve and the optimal up-slope is estimated. The weight for intensity point t is defined as its gradient magnitude computed from sᵢ′(t). A scatter interpolation strategy is finally applied to fill the ‘holes’ which often appear on the noisy background where the algorithm cannot find enough stable features across the scale space.

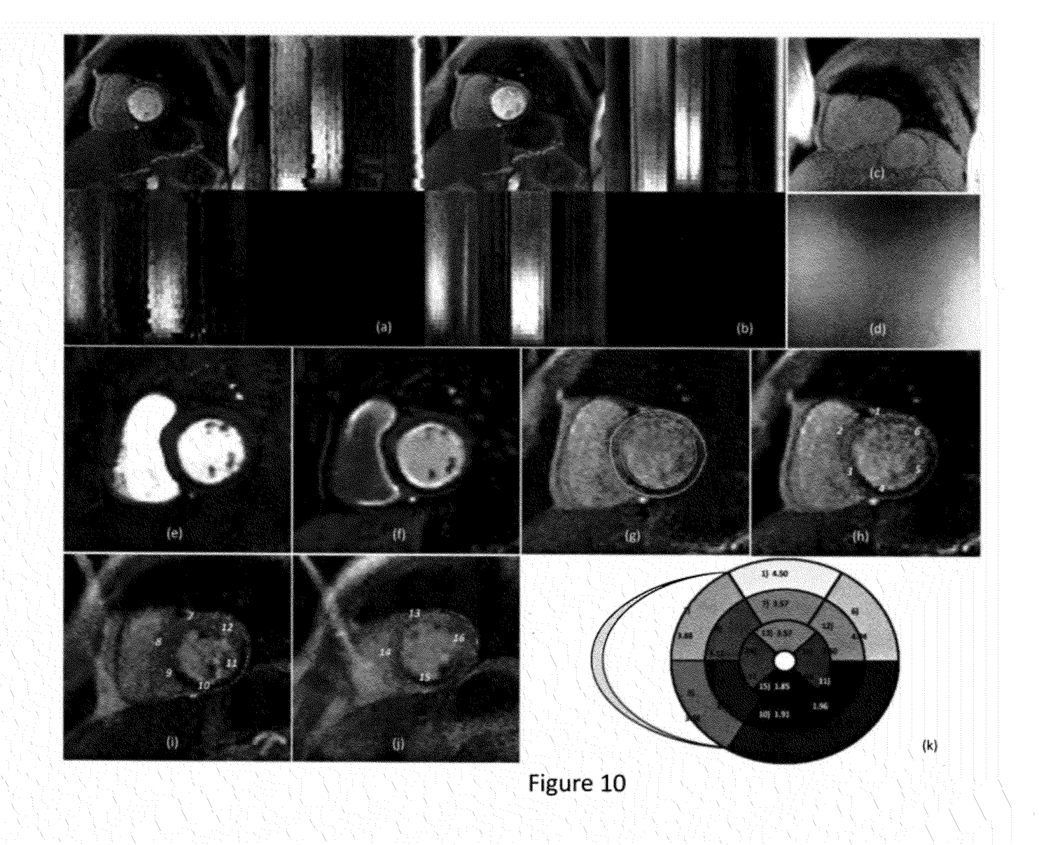

Once the method 100 computes the parameter map, the method 100 can perform segmental analysis by splitting the epicardium and endocardium contours according to the AHA model (Step 155). The left ventricle center and right ventricle insertion anterior point are used to determine the starting angle for each slice (e.g. basal, medial and apical). Pixels within each segment are counted to compute the average as the quantitative value for this segment. As a result, the AHA model can be computed in a fully automated manner as shown in FIG. 10. FIG. 10 shows the (a) original perfusion series; (b) the processed perfusion series after MOC, SCC and NOS; (c) the PD images; (d) the estimated inhomogeneity field; (e-f) the estimated perfusion parameters maps (e: upslope; f: area-under-curve); (g) the segmentation results with detection of the RV insertion and LV center points; (h) the per-segmental delineation; (i-j) the per-segmental delineation results for medial (i) and apical (j) slices; and (k) the AHA 16 model with averaged up-slope values.

Other modifications are possible within the scope of the invention. For example, the subject patient to be scanned may be a human subject, animal subject or any other suitable object. Also, although the steps of the methods 100, 400 have been described in a specific sequence, the order of the steps may be re-ordered in part or in whole and the steps may be modified, supplemented, or omitted as appropriate. Also, the methods 100, 400 may use various well known algorithms and software applications to implement the steps and substeps. Further, the methods 100, 400 may be implemented in a variety of algorithms and software applications. Further, the methods 100, 400 may be supplemented by additional steps or techniques. It is also understood that the methods 100, 400 may carry out all or any of the steps using real-time data, stored data from an image data archive or database 18, data from a remote computer network, or a mix of data sources.

Also, the various components of the imaging system 10 are conventional and well known components. They may be configured and interconnected in various ways as necessary or as desired. Further, although in the described methods 100, 400 the health professional may use self-contained imaging instrumentation and tools, the health professional may use other instrumentation or tools in combination with or in place of the imaging instrumentation and tools described for any step or all the steps of the methods 100, 400, including those that may be made available via telecommunication means. Further, the described methods 100, 400, or any steps, may be carried out automatically by appropriate imaging instrumentation and tools or with some manual intervention.

What is claimed is:

1. A method of dynamic MR perfusion imaging, comprising:
a. obtaining a series of perfusion images of a target cardiac area;
b. correcting imaging errors arising from cardiac motion;
c. correcting imaging errors arising from surface coil inhomogeneity;
d. segmenting the target cardiac area; and
e. generating perfusion parameters for the perfusion images that enable analysis of the segmented target cardiac area.

2. The method of claim 1, wherein the target cardiac area comprises the myocardium.

3. The method of claim 1, wherein the perfusion images are first pass perfusion images.

4. The method of claim 1, wherein the correcting imaging errors arising from cardiac motion comprises detecting a key-frame in the perfusion series.

5. The method of claim 4, wherein the key-frame defines a reference image and the correcting step comprises correcting the relative motion between the reference image and other phases.

6. The method of claim 5, wherein the key-frame comprises an image frame in which the target cardiac area has good contrast compared to the blood pool and surrounding tissues.

7. The method of claim 5, wherein the detecting step comprises computing the standard deviation of intensity image for the perfusion series and selecting the frame having similar contrast to the standard deviation of intensity image as the key-frame.

8. The method of claim 7, wherein the selecting step comprises computing cross correlation ratios (CC) between every phase in the perfusion series and the standard deviation of intensity image and selecting the phase corresponding to the maximal CC ratio as the key-frame.

9. The method of claim 4, wherein the correcting imaging errors arising from cardiac motion further comprises performing a registration of the key-frame with other images in the perfusion series.

10. The method of claim 9, wherein the performing step comprises performing consecutive motion compensation.

11. The method of claim 9, wherein the performing step comprises consecutively performing multiple 2D-2D registrations between temporally adjacent images.

12. The method of claim 9, wherein the performing step comprises applying a fast variational non-rigid algorithm to the perfusion series.

13. The method of claim 1, wherein the correcting imaging errors arising from surface coil inhomogeneity comprises estimating a smoothing surface coil inhomogeneity field from proton density (PD) images of the target cardiac area and applying the estimated field to all perfusion frames.

14. The method of claim 13, wherein the estimating step comprises acquiring the PD images before obtaining the perfusion series and estimating the surface coil inhomogeneity field via an approximation of B-Spline Free Form Deformation (FFD).

15. The method of claim 1, wherein the segmenting step comprises detecting selected cardiac landmarks.

16. The method of claim 1, wherein the segmenting step comprises detecting the center of the blood pool and the right ventricle insertion points as cardiac landmarks.

17. The method of claim 16, wherein the segmenting step further comprises segmenting the endocardium and the epicardium contours.

18. The method of claim 1, wherein generating perfusion parameters comprises estimating perfusion parameters for each pixel in a respective image.

19. The method of claim 17, further comprising splitting the epicardium and endocardium contours according to the American Heart Association (AHA) standards model for an analysis of the segmented target cardiac area.

20. A computer-assisted method of processing myocardial MR perfusion images, comprising correcting imaging errors arising from myocardial motion and B-1 field inhomogeneity; segmenting the myocardium images into standards-defined segments; and calculating perfusion measures that enable analysis of the segmented myocardium images.

21. The method of claim 20, wherein the correcting step comprises detecting a key-frame of the perfusion images and performing a registration between the key-frame and other phases of the perfusion images to correct the relative motion between the key-frame and a respective phase.

22. The method of claim 21, wherein the performing step comprises performing consecutive motion compensation on the perfusion images.

23. The method of claim 20, wherein the correcting step comprises applying an estimated smoothing B-1 field imhomogeneity field to all perfusion frames to correct for B-1 field coil inhomogeneity.

24. The method of claim 20, wherein the correcting step comprises estimating B-1 field-induced intensity variations in a respective image and generating an estimated B-1 field-induced intensity variation-compensated image.

25. The method of claim 20, wherein the segmenting step comprises detecting cardiac landmarks and segmenting the epicardium and endocardium contours.

26. The method of claim 25, wherein the segmenting the epicardium and endocardium contours comprises:
a. classifying the pixels in the perfusion images;
b. transforming the key-frame image into a polar image based on the detected cardiac landmarks;
c. computing gradients, in the polar space, for the recoveries of the epicardium and endocardium contours; and
d. recovering each of the epicardium and endocardium contours by finding the respective shortest path in the polar image.

27. The method of claim 26, wherein the classifying step comprises extracting the main gray level modes in the key-frame image.

28. The method of claim 26, wherein the transforming step comprises determining a polar coordinate system in which to transform the image by using the blood pool landmark as the center and the distance to the anterior right ventricle insertion point as a radius.

29. The method of claim 26, further comprising recovering the contours in a next slice by repeating steps a, b, and c; propagating the respective contours from the current slice to generate respective a priori contours for each of the epicardium and endocardium; and recovering each of the epicardium and endocardium contours by finding the respective shortest path in the polar image using the respective a priori contours.

30. The method of claim 20, wherein the calculating step comprises calculates perfusion parameter maps with estimated perfusion parameters for each pixel in an image.

31. The method of claim 20, wherein the calculating step comprises computing the American Heart Association (AHA) standards model for an analysis of the segmented myocardial images.

32. A system for dynamic MR perfusion imaging, comprising an MR imaging system having an imager that images the cardiac area to acquire image data and a processor that manipulates the acquired image data and stored image data to correct imaging errors arising from myocardial motion and B-1 field inhomogeneity; segment myocardium images into standards-defined segments; and calculating perfusion measures that enable analysis of the segmented myocardium images.
